(19) United States
(12) Patent Application Publication
(10) Pub. No.: US 2026/0151553 A1
(43) Pub. Date: Jun. 4, 2026

(54) METHOD SYSTEM AND APPARATUS FOR DRUG DELIVERY AND TREATMENT

(71) Applicant: ForCast Orthopedics, Inc., Denver, CO (US)

(72) Inventors: Peter D. NOYMER, Los Gatos, CA (US); Miguel GUZMAN, Hayward, CA (US); Reynaldo J. QUINTANA, Menlo Park, CA (US)

(73) Assignee: ForCast Orthopedics, Inc., Denver, CO (US)

(21) Appl. No.: 19/457,387

(22) Filed: Jan. 23, 2026

Related U.S. Application Data
(63) Continuation of application No. PCT/US2024/039519, filed on Jul. 25, 2024.
(60) Provisional application No. 63/515,599, filed on Jul. 26, 2023.

(51) Int. Cl. A61M 5/14; A61K 38/14; A61K 47/10; A61K 47/26

(52) U.S. Cl. CPC A61M 5/1409; A61K 38/14; A61K 47/10; A61K 47/26; A61M 5/1413; A61M 2202/064

(57) ABSTRACT

A method, system, and apparatus for drug delivery and treatment may be provided. The drug delivery method may be used to treat health conditions such as periprosthetic joint infections (PJI) and other orthopedic joint infections. The drug delivery method and apparatus may include a fluid delivery mechanism, which may include a fluid reservoir which may be filled with a high-concentration antibiotic formulation. The high-concentration antibiotic formulation may include an antibiotic concentration ranging from about 10 mg/mL to about 200 mg/mL may be dissolved in an aqueous solution, and then may be stored in refrigerated or frozen conditions. The fluid from the reservoir may be delivered, using a medical device or instrument, in discrete doses for bolus administration.

### BACKGROUND

For many diseases patients are cured more effectively if the therapeutic agent is administered in a manner that preferentially achieves a therapeutic level in the anatomical region of interest. For example, treating patients with periprosthetic joint infection (PJI) can be more effective by delivering the antibiotic dose as a bolus to the infected joint cavity rather than through intravenous (IV) administration. However, such formulation needs to have high concentration of the antibiotic drug substance to minimize the dosing volume while remaining physically and chemically stable for a period required for storage and administration to patient.

Under the current standard of care, a reconstituted vancomycin solution is typically administered to a PJI patient through IV administration, which puts the drug directly into the systemic circulation. As a result, only a small fraction of the administered antibiotic reaches the periprosthetic joint space, potentially not reaching therapeutic levels at the site of the infection. Another method of medication delivery is through antibiotic-laden prosthetic cements or materials, which can deliver a high dose to the infected joint but only for a very short period of time. Not only are both of these approaches (IV administration and antibiotic-laden materials) potentially ineffective in clearing the infection due to being subtherapeutic, but they also increase the risk of antibiotic resistance in the infecting pathogen, thereby making it even more difficult to treat and eventually eliminate the infection.

### SUMMARY

Exemplary embodiments described herein generally relate to a fluid delivery device and a fluid delivery method to treat orthopedic joint infections, and to a fluid reservoir for delivering a high-concentration medicinal formulation into an infected joint area, and a method for preparing high-concentration medicinal formulations.

Such a fluid delivery device may include: a fluid reservoir filled with a high-concentration medicinal formulation. In an exemplary embodiment, the high-concentration formulation may have a concentration of about 10 mg/mL to about 200 mg/mL of an antibiotic solute, such as vancomycin hydrochloride, tobramycin, gentamicin, rifampin, daptomycin, ceftriaxone, lineozid, or cefazolin, dissolved in an aqueous solvent, such as about 5% dextrose solution in water (D5W), buffered saline, about 10% ethanol aqueous solution or a normal saline solution. In some embodiments, the formulation may include an anti-microbial preservative such as phenol, benzyl alcohol, phenoxyethanol, methylparaben and propylparaben, or a combination of several anti-microbial preservatives. This high-concentration antibiotic formulation may in some embodiments be stored under refrigerated or frozen conditions to maintain physical and chemical stability. The refrigerated conditions may be, for example, 2-5° C. Also, in an exemplary embodiment, the fluid reservoir may be able to be attached to a medical implant or other medical device, such as an infusion pump and/or catheter, for daily administration.

### BRIEF DESCRIPTION OF THE FIGURES

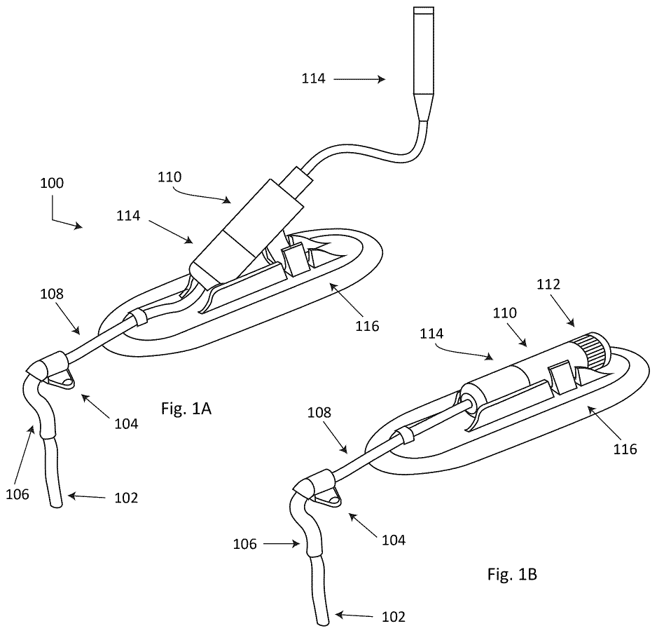

Exemplary FIG. 1A shows the operational position of an alternative embodiment of a fluid delivery device.

Exemplary FIG. 1B shows of the storage position of an alternative embodiment of a fluid delivery device.

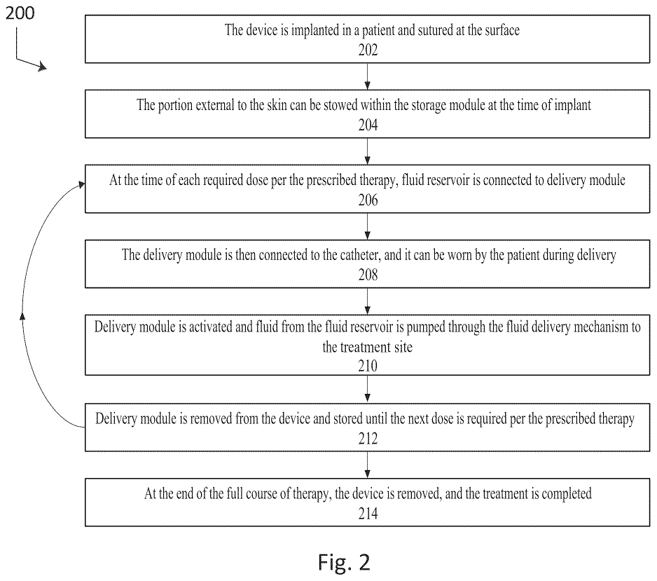

Exemplary FIG. 2 shows an embodiment of a fluid delivery method using a fluid delivery device.

### DETAILED DESCRIPTION

Aspects of the invention are disclosed in the following descriptions directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

In one or more exemplary embodiments a drug delivery method and apparatus may be provided.

In an exemplary embodiment the drug delivery method and apparatus may be used to treat, for example, periprosthetic joint infections (PJI) or other orthopedic infectious-disease conditions, for example osteomyelitis, septic arthritis, or fracture-related infections. It may be understood that in some embodiments the drug delivery method and apparatus may be used to treat other conditions such as implant-related infections and other joint conditions. The drug delivery method and apparatus may include a fluid delivery mechanism, which may be used to administer fluid to an infected joint through the intra-articular (IA) space or other portion of the body. In some embodiments, the fluid delivery mechanism may include a fluid reservoir, which may include a syringe or cartridge. In some embodiments, the filled reservoir may contain a high concentration antibiotic formulation. In other embodiments, the fluid reservoir may contain other fluids, for example, saline or hyaluronic acid flush, different fluids with clinical benefit, or a combination of fluids such as a mixture of saline, hyaluronic acid, local anesthetics, steroids, and antibiotics. In some embodiments, the fluid reservoir may deliver fluid to an infected area through a fluid delivery mechanism. In some embodiments the delivery mechanism may include an internal delivery mechanism, such as an infusion pump, which may be connected to an external delivery mechanism, such as a medical implant or other medical device worn by a patient. In still other embodiments the fluid reservoir may be able to deliver fluid to an infected area in another way.

In one or more exemplary embodiments a method for preparing high-concentration antibiotic formulations may be provided. A high-concentration antibiotic formulation for use in the fluid delivery device may include an antibiotic concentration ranging from about 10 mg/mL to about 200 mg/mL dissolved in an aqueous solution and, in some embodiments, may be stored in refrigerated or frozen conditions. Some embodiments may include a diluent of about 5% dextrose aqueous solution (D5W). Other embodiments may include an about 10% ethanol aqueous solution. Still other embodiments may include a standard saline solution or a buffered saline solution. In an exemplary embodiment, the high-concentration antibiotic formulation may have a vancomycin hydrochloride formulation, which may have a concentration of about 50 mg/mL to about 100 mg/mL, may utilize a diluent such as about D5W, and may be stored under refrigerated or frozen conditions to extend the shelf life, for example, up to 4 weeks or more. In other embodiments, other antibiotics, such as tobramycin, gentamicin, rifampin, and cefazolin, may be used in similar conditions.

An exemplary method of using the fluid delivery device may be provided. The drug delivery method may include a fluid delivery mechanism, which may include a fluid reservoir with a high-concentration antibiotic formulation, or other medicinal fluid. In an exemplary embodiment, the fluid may be prefilled. In an exemplary embodiment, the fluid reservoir may be a cartridge or syringe. The process of prefilling the fluid reservoir with the high-concentration antibiotic formulation or other fluid may be performed by, for example, a manufacturer or a specialty pharmacy. In a next step, the prefilled fluid reservoir may be delivered to a medical professional or directly to a patient. In a next step, a patient or medical professional may connect the prefilled fluid reservoir to a fluid delivery mechanism, for example, a medical implant or other medical device or instrument, such as an infusion pump, which may be worn by the patient.

In an exemplary embodiment, an antibiotic or other solvent may be stored as lyophilized powders due to relatively short shelf life when reconstituted in an aqueous solution. In these cases, the fluid reservoir may contain two or more chambers with a membrane/barrier between the chambers. In some embodiments one or more chambers may contain a powder while one or more other chambers contain a diluent, the barrier may be breakable by the patient or a nurse/other medical professional which may allow the powder to combine with the diluent to create a medicinal fluid.

In an exemplary embodiment, the fluid reservoir may be manufactured with a breakable membrane/barrier that separates a lyophilized powder from a diluent. In this embodiment, the fluid delivery method may include an intermediary step of reconstituting the solution by breaking the barrier and mixing the lyophilized powder with the diluent.

In a next step, fluid from the fluid delivery device may be administered through the fluid delivery mechanism to the treatment site. In a next step, the intended dose of fluid may be delivered through bolus administration, which may be understood to mean a discrete amount of the fluid may be administered within a specific time frame. The bolus dose may be considered fully delivered when the fluid reservoir is emptied, when a predetermined amount of fluid has been pumped, and/or when a specified amount of time has passed. In some embodiments, the fluid reservoir may be replaced. In some embodiments, some or all of these steps may be repeated periodically, for example once or twice per day, or once every other day, until treatment is finished. In a last step, the treatment may be finished, and the fluid delivery mechanism may be removed, for example.

Referring to FIGS. 1A-1B, an embodiment of the fluid delivery device 100 in an operational position 150 and an embodiment in a storage position 160 may be shown and described. The fluid delivery device 100 may include an internal delivery mechanism 102 which may be implanted into a patient and connect to, for example, an infected joint space. It may be understood that when in use the internal delivery mechanism 102 may be placed such that it is inside a patient. The internal delivery mechanism 102 may connect with a first side of a pivot point 104, which may be understood to be at the skin surface of a patient. The pivot point 104 may further be connected to a cuff 106 which may cover a portion of the internal delivery mechanism 102, for example the cuff 106 may be about 15 to 30 mm long. It may be understood that the cuff 106 may allow for tissue ingrowth. A second side of the pivot point 104 may further connect to an external delivery mechanism 108 which may feed into a delivery module 110. It may be understood that in some embodiments the internal delivery mechanism 102 and external delivery mechanism 108 may be two parts of the same catheter piece that continuously runs through the pivot point 104, while in other embodiments the internal delivery mechanism 102 and external delivery mechanism 108 may be separate mechanisms that are attached to each side of the pivot point 104 in a fluid tight fashion, for example being glued. The delivery module 110 may include, for example, a cap 112 that may protect the site when not in use and an infusion line connector, which may be a Luer-lock connector in an embodiment. In some embodiments the cap 112 may include a magnet and/or lock threads, which may secure the cap 112 to the fluid reservoir 114. The delivery module 110 may be attached or held to the patient, for example by an adhesive patch 116, which may be a pressure sensitive adhesive patch. The delivery module 110 may be attached to the adhesive patch 116 via, for example, hook and loop pads or patches. The fluid reservoir 114 may also be attached or held to the patient in a similar manner. In some embodiments the cap 112 may be tethered to the fluid reservoir 114 to prevent loss.

It may be understood that in some embodiments the internal delivery mechanism 102 and external delivery mechanism 108 may be a single delivery mechanism that passes from being implanted in the patient to external the patient via the pivot point 104. In an embodiment the delivery mechanism may be a continuous pliable tube and may have a diameter of about 1.0 to 3.0 mm, for example, a catheter. The cuff 106 may be made from a material that promoted skin and would healing and provides adhesion to enhance skin closure around it, which may be understood to decrease the risk of infections. In some embodiments the cuff 106 may be made from, for example but not limited to, polyester materials such as Dacron, and/or metal mesh materials such as titanium or stainless steel. In other embodiments the cuff 106 may instead be a polymer extrusion.

The pivot point 104 may ensure the internal delivery mechanism exits the body with a tight bend, which may prevent the internal and external delivery mechanisms from accidental snags and/or kinking. The pivot point 104 may further be configured to maximize the exposure area of the cuff 106, which may allow for easier cleaning and disinfecting of the cuff 106. The pivot point 104 may further have two or more suture points to secure it to the skin at the time the internal delivery mechanism 102 is implanted, may be designed to be split and readily removed from the internal delivery mechanism 102 in order to facilitate ease of removal at the end of treatment. It may be understood that once the pivot point 104 is removed a coring tool may be utilized to cut the skin and soft tissue around the cuff 106 and placed over the catheter tube in order to fully remove the fluid delivery device 100.

The delivery module 110 may further contain a check valve that may prevent fluid backflow. The check valve may mitigate compound, saline, or body fluids from exiting the patient through the infusion line connector during connection, removal, or non-use of the infusion line. In some embodiments the delivery module 110 may have an operational position 150, and a storage position 160. In the storage position 160 the delivery module 110 may collapse into and be secured by the fluid reservoir 114, which may decrease the profile of the delivery module 110 and prevent snagging or catching while not in use. In some embodiments the delivery module 110 may be a single use disposable module, while in other embodiments the module may be multi-use and/or refillable.

The fluid reservoir 114 may mitigate fluid loss out of the catheter when the infusion line is not connected, for example via the cap 112, check valve, or both. In some embodiments the cap 112 may provide visual or tactile feedback when properly placed, for example through the use of a magnet. In some embodiments, a portion of the external delivery mechanism 108 may be further secured a few cm away from the pivot point 104, which may provide strain relief, and in conjunction with sutures to the skin may provide strain relief to the delivery mechanism as it enters the body.

It may be understood that the fluid delivery device 100 may be connected to an external delivery mechanism 108 and may be utilized to deliver treatment to an area interior to a patient, for example a washout treatment that includes cleaning an infected joint or other area without requiring removal of prosthetic hardware and/or a revision treatment such as cleaning and removal or replacement of prosthetic hardware. It may be understood the connections between the fluid delivery device, infusion line, and any external structure (such as a delivery module) may be liquid tight.

Referring now to FIG. 2, an embodiment of a method for a fluid delivery system using, for example, the fluid delivery device 100 may be shown.

In a first step 202, the fluid delivery device 100 may be implanted in the patient and sutured at the surface. The fluid delivery device may be positioned such that a portion of the device is implanted in the patient and a portion of the device is external to the patient's skin. In a next step 204, an external delivery mechanism 108 may be stowed within a storage module at the time of implant. In a next step 206 at the time a dose is required by the patient, the fluid reservoir 114 may be connected to the delivery module 110. In a next step 208, the delivery module 110 may connect to the external delivery mechanism 108, and the delivery module 110 may be worn by the patient during fluid delivery. In a next step 210, the delivery module 110 may be activated and fluid from the fluid reservoir 114 may be pumped through the delivery mechanism to the treatment site. In a next step 212, once pumping is finished (i.e., which may be when the fluid reservoir 114 is emptied, when a predetermined amount of fluid has been pumped, and/or when a specified amount of time has passed), delivery module 110 may be removed from the external delivery mechanism 108 and stored. In some embodiments, steps 206-212 may be repeated periodically (e.g., once per a day, or once every other day) until treatment is finished. In a last step 214, treatment may be finished after, for example a full course of therapy; after a specified period of time (e.g., 6 weeks, 2 months), or after the initial problem (e.g., PJI) has been cured or sufficiently treated. In some embodiments a checkup may be performed after a specified period of time in order to determine whether treatment should continue. It may be understood that after treatment, additional surgeries may be performed and the fluid delivery device 100 may be removed. An external fluid delivery mechanism 108 may be the portion of the fluid delivery device 100 external to the patient's skin.

The foregoing descriptions illustrate the principles, preferred embodiments, and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A fluid delivery device comprising:
a prefilled fluid reservoir;
a high concentration medicinal fluid stored within the fluid reservoir;
a delivery module that facilitates the delivery of the fluid from the reservoir to the anatomical region requiring the treatment through a delivery mechanism;
an external delivery mechanism that connects to a pivot point and is external to the patient's skin;
an internal delivery mechanism that delivers the high concentration medicinal fluid directly to an affected area in a bolus dosage;
a pivot point that connects the internal and external delivery mechanisms;
a cuff that is made of material that promotes skin and wound healing.

2. The fluid delivery device of claim 1, wherein the high concentration medicinal solution comprising:
an antibiotic at a concentration of 10-200 mg/mL; and
an aqueous solvent used as a diluent.

3. The drug delivery system of claim 2, wherein the antibiotic is vancomycin hydrochloride at a concentration of 50-100 mg/mL; and
the diluent is a solution of 5% dextrose.

4. The fluid delivery device of claim 1, wherein the formulation also includes an anti-microbial preservative.

5. The fluid delivery device of claim 1, wherein the pivot point is designed to be split and readily removed from the internal delivery mechanism.

6. The fluid delivery device of claim 1, wherein the pivot point has a plurality of suture points to secure it to the skin.

7. The fluid delivery system of claim 1, wherein the bolus dosage administration is repeated on a periodic basis throughout the course of a treatment.

8. The fluid delivery system of claim 1, wherein the bolus dosage is administered by a medical professional.

9. A fluid delivery method comprising:
preparing a high concentration medical solution;
preserving the solution by storing the fluid under refrigerated or frozen conditions;
prefilling a fluid reservoir with the high concentration medicinal fluid;
connecting the prefilled fluid reservoir to a delivery module;
pumping the fluid into an external delivery mechanism using the delivery module;
transferring the fluid from the external delivery mechanism to the internal delivery mechanism through a pivot point;
transporting the fluid below the skin barrier in an internal delivery mechanism;
administering the medication in a bolus dosage from the internal delivery mechanism directly to an affected area;
promoting skin and wound healing with a cuff disposed on the internal delivery mechanism.

10. The fluid delivery method of claim 9, wherein preparation of the medical solution comprises:
reconstituting an antibiotic solution to a concentration of 10-200 mg/mL;
diluting and dissolving the antibiotic with an aqueous solvent.

11. The fluid delivery method of claim 10, wherein reconstituting the antibiotic solution involves diluting vancomycin hydrochloride with 5% dextrose solution to a concentration of 50-100 mg/mL.

12. The fluid delivery method of claim 9, wherein preparation of the medicinal solution includes the adding an anti-microbial preservative.

13. The fluid delivery method of claim 9, wherein splitting and removing the pivot point can be done to facilitate the fluid delivery.

14. The fluid delivery method of claim 9, wherein suturing the pivot point is done at one or more points to secure the pivot point to the skin.

15. The fluid delivery method of claim 9, wherein the administration of the bolus dosage is performed by a medical professional.

16. The fluid delivery method of claim 9, wherein the administration of the bolus dosage is repeated periodically over the course of treatment.

17. The fluid delivery method of claim 9, wherein the administration of the bolus dosage is performed by a medical professional.
